(19) United States
(12) Patent Application Publication
(10) Pub. No.: US 2015/0157681 A1
(43) Pub. Date: Jun. 11, 2015

(54) METHOD OF USING TRAUMEEL IN ORAL SURGICAL PROCEDURES

(71) Applicant: Walter Tatch, Northbrook, IL (US)

(72) Inventor: Walter Tatch, Northbrook, IL (US)

(21) Appl. No.: 14/097,317

(22) Filed: Dec. 5, 2013

(51) Int. Cl. A61K 36/81; A61K 36/28; A61K 36/30; A61K 33/14; A61K 33/28; A61K 36/714; A61K 36/185

(52) U.S. Cl. CPC A61K 36/81; A61K 36/714; A61K 36/28; A61K 36/185; A61K 33/14; A61K 33/28; A61K 36/30

(57) ABSTRACT

Methods of reducing inflammation, pain and bruising after oral surgery with intramucosal delivery of Traumeel are disclosed.

### BACKGROUND OF THE INVENTION

1. Field of the Invention

The present invention relates generally to dosage forms and methods of using dosage forms of Traumeel in surgical procedures.

2. Background

Traumeel is a fixed combination of diluted plant and mineral extracts. The combination is currently used to treat acute musculoskeletal injuries, such as sprains and traumatic injuries, and as supportive therapy in pain and inflammation of the musculoskeletal system. It can be used in the form of tablets, drops, injection solution, ointment, and gel. (Schneider. Traumeel—an emerging option to nonsteroidal anti-inflammatory drugs in the management of acute musculoskeletal injuries. International Journal of General Medicine 2011:4 225-234).

For example U.S. Patent Application No. 2012/0070507 A1 to Howell et al. teaches Traumeel compositions and methods for the treatment of soft tissue injury, sports-related injury, physical overuse of muscles, ligaments or joints, and trauma, traumatic stress or sudden jolts on bones and tissues in an individual and is incorporated herein by reference in its entirety.

Farrer. A Homeopathic Answer to Post Operative Inflammation. ViewPoint: Dental Probe Magazine October 2012 teaches that deep scaling procedures, periodontitis and suppurative periodontal pockets can all benefit from the subcutaneous injection of Traumeel and further suggests the use of Traumeel supplements after implant surgery and is incorporated herein by reference in its entirety.

Zilinskas et al. Inhibition of peripheral blood neutrophil oxidative burst in periodontitis patients with a homeopathic medication Traumeel S. Med Sci Monit, 2011; 17(5): CR284-291, incorporated herein by reference in its entirety, evaluates the effects of Traumeel S on oxidative function of peripheral blood neutrophils of periodontitis patients and controls in vitro.

Inflammation and pain are common complications associated with oral surgery. These side effects along with bruising, trismus and edema affect the quality of a patients' life after the surgery. Techniques to reduce swelling, pain and inflammation are available, but have certain drawbacks including negative reactions to certain types of analgesics. In particular, some patients may be allergic to NSAIDS. Different techniques for the management of pain and inflammation include corticosteroids, drains and analgesics such as NSAIDs (Osunde et al. Management of inflammatory complications in third molar surgery: A review of the literature. Afr Health Sci. 2011 September; 11(3): 530-537). However, this review showed that no single modality of management effectively prevents the occurrence of these complications without undesirable side effects.

Therefore, there is a need in the art to promote anti-inflammatory, pain reducing and wound healing effects after surgery and particularly after oral surgery.

### BRIEF SUMMARY OF THE INVENTION

Described herein is a novel application of Traumeel in an injectable form.

In one embodiment, the invention is directed to a method of reducing or ameliorating pain, inflammation or bruising at a surgical site comprising injecting Traumeel at the surgical site wherein the Traumeel is injected in the mucosal layer of the skin. Preferably, the mucosal layer of the skin is located in the oral or buccal cavity. In one instance the Traumeel injection is given post-operatively. In another instance, the Traumeel injection is given intra-operatively.

In one embodiment, the invention is directed to a method of reducing or ameliorating pain, inflammation or bruising at a surgical site comprising injecting Traumeel at the surgical site wherein the dosage unit of Traumeel is from 0.5 to 2 cc.

In one embodiment, the invention is directed to a method of reducing or ameliorating pain, inflammation or bruising at a surgical site comprising injecting Traumeel at the surgical site wherein the dosage unit of Traumeel is from 1 to 1.5 cc.

In one embodiment, the invention is directed to a method of reducing or ameliorating pain, inflammation or bruising at a surgical site comprising injecting a dosage unit of Traumeel wherein the dosage is from 0.5 to 2 cc and such dosage unit is administered once for every 0.5-3 cm² area of the surgical site.

In one embodiment, the invention is directed to a method of reducing or ameliorating pain, inflammation or bruising at a surgical site comprising injecting Traumeel at the surgical site wherein the surgical site is in the oral cavity.

In one embodiment, the invention is directed to a method of reducing or ameliorating pain, inflammation or bruising at a surgical site comprising injecting Traumeel at the surgical site wherein the surgical site is the result of a surgery selected from the group consisting of: wisdom teeth removal, dental implants placement, advanced bone reconstruction, the use of BMP (bone morphogenetic proteins) for bone grafting, dental extractions, bone grafting, soft tissue grafting, facial trauma, facial reconstruction, orthognathic surgery, periapical surgery and any other oral surgical procedure.

Advanced bone reconstruction includes the reconstruction of maxillofacial defects after oncologic ablative surgery, trauma, or as the result of congenital anomalies. Such surgery can be very painful and result in severe swelling and pain. Performing these surgeries to reconstruct maxillofacial defects can aid in a patient's ability to speak and eat. However, the inflammation and pain as a result of these surgeries can negatively affect a subjects post-operative quality of life. (Kademani et al. Reconstruction of Head and Neck Defects: A Systematic Approach to Treatment. SEMINARS IN PLASTIC SURGERY/VOLUME 22, NUMBER 3 2008 pp 141-155).

The application of bone morphogenetic protein to cure osseous defects has been encouraging. Bone graft substitute materials are also used to cure congenital defects such as cleft palate, facial clefts, and facial asymmetry and the use of such results in post-surgical swelling, inflammation and pain. (Boyne. Application of bone morphogenetic proteins in the treatment of clinical oral and maxillofacial osseous defects. J Bone Joint Surg Am. 2001; 83-A Suppl 1(Pt 2):S146-50).

Periapical surgery is associated with inflammation as part of the wound healing process (Lin et al. Guided Tissue Regeneration in Periapical Surgery. J Endod 2010; 36:618-625). Again, this inflammation interrupts a patients normal daily activities such where movement of the mouth or jaw is required.

In one embodiment, the invention is directed to a method of reducing or ameliorating pain, inflammation or bruising at a surgical site comprising injecting Traumeel at the surgical site wherein one or more additional therapeutic agents are administered before, during and/or after a surgical procedure and the additional therapeutic agents are agents known to reduce pain, inflammation and/or bruising.

In one embodiment, the invention is directed to a method of reducing the time to maximum swelling around a surgical site, comprising post-operatively injecting Traumeel at the surgical site. In one instance the Traumeel injection is given post-operatively. In another instance, the Traumeel injection is given intra-operatively.

In one embodiment, the invention is directed to a method of reducing the time to maximum swelling around a surgical site, wherein the Traumeel is injected in the mucosal layer. In one instance the Traumeel injection is given post-operatively. In another instance, the Traumeel injection is given intra-operatively.

In one embodiment, the invention is directed to a method of reducing the time to maximum swelling around a surgical site, wherein the dosage unit of Traumeel is from 0.5 to 2 cc.

In one embodiment, the invention is directed to a method of reducing the time to maximum swelling around a surgical site, wherein the dosage unit of Traumeel is from 1 to 1.5 cc.

In one embodiment, the invention is directed to a method of reducing the time to maximum swelling around a surgical site, wherein the surgical site is in the oral cavity.

In one embodiment, the invention is directed to a method of reducing the total inflammatory response time at a surgical site post-operatively comprising post-operatively injecting Traumeel at the surgical site wherein the Traumeel is injected in the mucosal layer of the epidermis. In one instance the Traumeel injection is given post-operatively. In another instance, the Traumeel injection is given intra-operatively.

In one embodiment, the invention is directed to a method of reducing the total inflammatory response time at a surgical site post-operatively comprising post-operatively injecting Traumeel at the surgical site wherein the dosage unit of Traumeel is from 0.5 to 2 cc.

In one embodiment, the invention is directed to a method of reducing the total inflammatory response time at a surgical site post-operatively comprising post-operatively injecting Traumeel at the surgical site wherein the dosage unit of Traumeel is from 1 to 1.5 cc.

In one embodiment, the invention is directed to a method of reducing the total inflammatory response time at a surgical site post-operatively comprising injecting Traumeel at the surgical site wherein the surgical site is in the oral cavity. In one instance the Traumeel injection is given post-operatively. In another instance, the Traumeel injection is given intra-operatively.

In one embodiment, the invention is directed to a method of inhibiting pro-inflammatory cytokines while stimulating anti-inflammatory cytokines at a surgical site comprising injecting Traumeel at the surgical site wherein the Traumeel is injected into the mucosal layer of the epidermis. In one instance the Traumeel injection is given post-operatively. In another instance, the Traumeel injection is given intra-operatively.

In one embodiment, the invention is directed to a method of inhibiting pro-inflammatory cytokines while stimulating anti-inflammatory cytokines at a surgical site wherein the dosage unit of Traumeel is from 0.5 to 2 cc.

In one embodiment, the invention is directed to a method of treating TMJ (Temporomandibular Joint Disorders) or TMD (Temporomandibular Disorders) comprising injecting 1 to 1.5 cc of Traumeel into the Temporomandibular joint.

In one embodiment, the invention is directed to a dosage unit form of Traumeel, wherein the dosage unit is from 0.5 to 2 cc and is intended for injection into a mucosal layer.

### BRIEF DESCRIPTION OF THE DRAWINGS

FIG. 1 is the clinical result of a sinus lift at 24 hours post-operative.

FIG. 2 is the clinical result of a sinus lift at 48 hours post-operative.

FIG. 3 is the clinical result of a sinus lift at 72 hours post-operative.

FIG. 4 shows a patient undergoing multiple implants placement.

FIG. 5 shows a 1.1 cc of Traumeel injected in the submucosal fashion on the right side and 1.1 cc on the left side of a surgical area after undergoing multiple implants placement.

Picture 6 shows a patient 24 hours after undergoing multiple implants placement surgery wherein he is exhibiting mild to moderate swelling around the chin and lower jaw areas.

FIG. 7 shows a patient 48 hours post-op, undergoing multiple implants placement, and shows his swelling has almost completely resolved (his swelling is 75-80% resolved).

FIG. 8 shows a lower right full bony impacted wisdom tooth.

FIG. 9, taken intra-operative, shows the surgical removal of an impacted wisdom tooth required of a full thickness mucoperiosteal flap and partial removal of bone.

FIG. 10, shows 1 cc of Traumeel injected intramucosally over the surgical site after the removal of an impacted wisdom tooth.

FIG. 11 shows the patient 24 hours post-op after the removal of an impacted wisdom tooth.

FIG. 12 shows the patient's swelling and bruising 48 hours after the removal of an impacted wisdom tooth.

FIG. 13 shows the patient's swelling and bruising 48 hours after the removal of an impacted wisdom tooth.

FIG. 14 shows a patient 72 hours post-op after the removal of an impacted wisdom tooth.

FIG. 15 shows extensive bone grafting performed in the anterior maxilla, extending to the posterior maxillary region.

FIG. 16 shows a total of 2 cc of Traumeel injected after a bone grafting procedure.

FIG. 17 shows a patient 24 hours after a bone grafting procedure.

FIG. 18 shows a patient 48 hours after a bone grafting procedure.

FIG. 19 shows a patient 72 hours after a bone grafting procedure.

### DETAILED DESCRIPTION OF THE INVENTION

The present invention will now be described more fully hereinafter with reference to the accompanying figures, in which preferred embodiments of the invention are illustrated. This invention may, however, be embodied in different forms and should not be construed as limited to the embodiments set forth herein. Rather, these embodiments are provided so that this disclosure will be thorough and complete, and will fully convey the scope of the invention to those skilled in the art.

The present invention is directed to a method of decreasing or reducing post-operative inflammation, edema, bruising, pain and related symptoms such as trismus after bodily insult and preferably after oral surgery. In one instance, Traumeel is post surgically injected into the mucosal layer of the epidermis. Mucous membranes are organized into different layers such as the mucosa (epithelium, lamina propria and muscularis mucosa) and the submucosa (Meissner's plexus). Mucous tissue (or mucosa) lines various anatomic structures in the mammalian body, including the mouth or oral cavity, eyes, respiratory tract (alveoli, bronchi, oral cavity, larynx, nasal cavity, pharynx, trachea), gastrointestinal tract (esophagus, stomach, small and large intestine, rectum), and genitourinary tract (urethra, urinary bladder, uterus and vagina).

The oral mucosa is the mucous membrane lining the inside of the mouth and consists of stratified squamous epithelium termed oral epithelium and an underlying connective tissue termed lamina propria. IgA found therein has a role in protecting against microbes and is an immunological regulator (http://en.wikipedia.org/wiki/Immunoglobulin_A).

Oral mucosa heals differently than non-mucosal epidermis (Chen et al. Positional differences in the wound transcriptome of skin and oral mucosa. Chen et al. BMC Genomics 2010, 11:471). Oral mucosal tissues contains much less inflammatory potential than skin since they contain less cytokines involved in the inflammatory process. IL-1b and TNF-a, for example are reduced in oral mucosal tissue.

As used herein “mucosal layer” means any mucous membranes (or mucosae, mucosas, or mucosa) or mucosal tissue. An injection into the mucosal layer includes injection of Traumeel into mucous tissue, the mucosal layer, the mucosa (epithelium, lamina propria and muscularis mucosa), the submucosa (Meissner's plexus) or submucosa which is the area adjacent to the mucosa. The injection site can be an intramucosal site or a submucosal site. In a preferred embodiment, an injection into the mucosal layer includes injection of Traumeel into the mucosal layer of the oral cavity. In another preferred embodiment, intramucosal injection includes injection of Traumeel into the mucosal layer of the oral cavity.

Traumeel can be obtained from any reputable supplier. For example, Traumeel can be purchased online or from Biologische Heilmittel Heel GmbH.

A dosage unit of Traumeel can be from 0.5 to 2 cc. Preferably, the dosage unit is from 1.0 to 1.5 cc. For example, the dosage form can be 1.0, 1.2, 1.3, 1.4 or 1.5 cc. Also, the dosage form can be about or approximately 1.0, 1.2, 1.3, 1.4 or 1.5 cc such as amounts which may fall between 1.0 and 1.2, or 1.2 and 1.3, or 1.3 and 1.4, 1.4 and 1.5 cc. This range of dosage units is preferential because it was unexpectedly found that less than about 0.5 cc and more than about 2.0 cc did not produce the benefits similar to a dosage form between about 0.5 to about 2 cc. In some instances, more than 2 cc per surgical site resulted in no reduction of inflammation, pain or bruising. This finding shows that Traumeel is dose dependent when administered in the mucosal layer.

The term “about” or “approximately” means within an acceptable error range for the particular value as determined by one of ordinary skill in the art, which will depend in part on how the value is measured or determined, i.e., the limitations of the measurement system. For example, “about” with respect to the compositions can mean plus or minus a range of up to 20%, preferably up to 10%, more preferably up to 5%. Alternatively, particularly with respect to biological systems or processes, the term can mean within an order of magnitude, preferably within 5-fold, and more preferably within 2-fold, of a value. Where particular values are described in the application and claims, unless otherwise stated the term “about” means within an acceptable error range for the particular value. For example, when referring to a period of time, e.g., hours, the present values (±20%) are more applicable. Thus, 6 hours can be, e.g., 4.8 hours, 5.5 hours, 6.5 hours, 7.2 hours, as well as the usual 6 hours. Another example includes a period of time measured in days such that “about” a week can refer to 6 days or 7 days or 8 days or any time in between such as 6 days 12 hours.

A dosage unit can be administered at multiple locations at the surgical site. For example, 0.5 cc intramucosal injection of Traumeel can be administered immediately above the surgical site and 0.5 cc intramucosal injection of Traumeel can be administered immediately below the surgical site. Another non-limiting example includes the delivery of 0.3 cc of Traumeel at one location immediately adjacent the surgical site and 0.5 cc of Traumeel at another location immediately adjacent to the same surgical site and 0.6 cc of Traumeel at a third location immediately adjacent the surgical site. In other words, the Traumeel dosage unit can be administered in multiple locations around the surgical site as desired or considered best practice by the practitioner or one skilled in the art.

For large surgical areas or for surgical sites that have extended surgical insult or surgical incision, a dosage unit as described herein can be administered at multiple locations associated with the same surgical area. For example, 0.5 to 2 cc can be administered for every 0.5-3 cm² of the insult area. Preferably, 0.5 to 2 cc can be administered for every 1.0-2.5 cm² of the insult area. Most preferably, 0.5 to 2 cc can be administered for every 1.5-2 cm² of the insult area. Specifically, 1.0 to 1.5 cc can be administered for every 0.5-3 cm² of the insult area. Preferably, 1.0 to 1.5 cc can be administered for every 1.0-2.5 cm² of the insult area. Most preferably, 1.0 to 1.5 cc can be administered for every 1.5-2 cm² of the insult area. Without being bound by theory, injection of the Traumeel into the mucosal tissue in this fashion provides localized stability and activity of the Traumeel. Once injected, the Traumeel remains in the mucosal tissue to effectively reduce the inflammation, pain, and bruising. This stability is not seen with other types of injections of Traumeel, such as subcutaneous injections or injections that are less than and greater than the range of 0.5-2 ccs.

For large surgical areas, those areas with dimensions greater than 0.5-3 cm², each area having a dimension of 0.5-3 cm² can each accommodate a dosage unit. A dosage unit can be administered at multiple locations in each dimensional area of from 0.5-3 cm² at the surgical site. In other words, 0.5 to 2 cc of Traumeel can be injected into a 0.5-3 cm² area. Since a large surgical area may have an area larger than 0.5-3 cm², multiple dosage units may be necessary around the surgical area. As a non-limiting example, 0.5 cc intramucosal injection of Traumeel can be administered at a first location at one 0.5-3 cm² area at the surgical site and 0.5 cc intramucosal injection of Traumeel can be administered at a second location in the same one 0.5-3 cm² area. Another non-limiting example includes the delivery of 0.3 cc of Traumeel at a first location immediately adjacent a first surgical site and 0.5 cc of Traumeel at another location immediately adjacent to the same first surgical site and 0.6 cc of Traumeel at a third location immediately adjacent the same first surgical site such that all three injections are in the mucous layer and all three injections are within a first 0.5-3 cm² area. At this same first surgical site, a second 0.5-3 cm² area can accommodate a second dosage unit such that 0.5 cc intramucosal injection of Traumeel can be administered at a first location at this second 0.5-3 cm² area at the first surgical site and 0.5 cc intramucosal injection of Traumeel can be administered at a second location in the second 0.5-3 cm² area adjacent the first surgical site.

More than one surgical procedure can be done in an oral cavity at one time. For each surgical site, a dosage unit can be administered at each site. Each dosage unit can be delivered in one or more locations at the surgical site as described above.

Traumeel injections can be given before, during or after surgery or a surgical procedure. In one instance, a dosage unit of Traumeel is administered prior to a surgical procedure. In another instance, one dosage unit is administered intra-operatively. Preferably, one dosage unit of Traumeel is given immediately after a surgery or surgical procedure. One dosage unit of Traumeel can be given post-surgically and additional dosage units can be administered on consecutive days following a surgical procedure. For example, a dosage unit can be administered on day 1 after a surgical procedure or on day 2 after a surgical procedure or day 3 after a surgical procedure or day 4 after a surgical procedure or day 5 after a surgical procedure or day 6 after a surgical procedure or day 7 after a surgical procedure. Such a dosage unit can be administered during a surgical procedure and subsequently can be administered as needed or as considered necessary by the administering professional. For example, a dosage unit can be administered immediately after a bone grafting procedure, for example, and then a second dosage unit can be administered on day 1 after the bone grafting procedure. An additional dosage can be administered on day 2 after the bone grafting procedure. An additional dosage can be administered on day 3 after the bone grafting procedure. An additional dosage can be administered on day 4 after the bone grafting procedure. An additional dosage can be administered on day 5 after the bone grafting procedure. The dosage can be administered based on the recommendation of one of ordinary skill in the art.

Traumeel can be administered immediately after surgery. When referring to timing, “immediately” has its ordinary meaning and means without delay. Immediate post-operative administration of Traumeel can include the administration as soon as the administering practitioner is able to administer the Traumeel. Immediate post-operative administration of Traumeel can also include the administration of Traumeel at the end of the surgical procedure. Immediate post-operative administration of Traumeel can further include the administration before the patient is released from the surgical suit. As it refers to proximity, “immediately” has its ordinary meaning and includes a location close to a surgical site. In some instances, the area immediate to or immediately adjacent the surgical site includes any area wherein the injection of Traumeel is therapeutically effective as described herein. Determination of a therapeutically effective amount is well within the capability of those skilled in the art, especially in light of the detailed disclosure provided herein. The size of the surgical site can vary depending on the surgical procedure and to that end, the area immediate to or immediately adjacent the surgical site can vary.

Traumeel can be injected at the surgical site. The term “at the surgical site” has its ordinary meaning and includes any surgical area and any area at or around the surgical site such that the Traumeel injection maintains its benefits of reducing inflammation, edema, swelling, pain and bruising for example. In some instances, the area at the surgical site includes any area wherein the injection of Traumeel is therapeutically effective. Determination of a therapeutically effective amount is well within the capability of those skilled in the art, especially in light of the detailed disclosure provided herein. The size of the surgical site can vary depending on the surgical procedure and to that end, the area around the surgical site can vary. In some instances, the surgical site is large and the area around the surgical site is commensurate in scope and size as the surgical site itself. In such a case, the area for effective treatment and effective dosing of Traumeel is larger. The therapeutically effective area at and around the surgical site will be appreciated by one of ordinary skill in the art.

The invention described herein includes an injectable dosage form for injection in the mucosal tissue to reduce inflammation, swelling, bruising and/or pain. The injectable dosage form can be prepared as a kit. The kit may include the dosage form and may further comprise instructions concerning use of the injectable dosage form. The instructions may comprise a warning, dosage regimen instruction, or any other information of value to the user. The instructions physically may comprise, for example, a separate instruction pamphlet, a paper, a placard, and/or one or more notices printed on a container such as a box that holds the unit doses. The instructions also may be supplied as a compact disk or other computer readable medium, or a video cassette.

The packaging for the kit can be any suitable packaging and may contain one or multiple dosage units in one package. The Traumeel composition will be contained in any suitable container capable of holding and dispensing the dosage form and which will not significantly interact with the composition and will further be in physical relation with the appropriate labeling. The labeling instructions will be consistent with the methods of treatment as described hereinbefore. The labeling may be associated with the container by any means that maintain a physical proximity of the two, by way of non-limiting example, they may both be contained in a packaging material such as a box or plastic shrink wrap or may be associated with the instructions being bonded to the container such as with glue that does not obscure the labeling instructions or other bonding or holding means.

The packaged dosage unit of Traumeel can be from 0.5 to 2 cc as described herein. Preferably, the dosage unit is from 1.0 to 1.5 cc. For example, the dosage form can be 1.0, 1.2, 1.3, 1.4 or 1.5 cc. Also, the dosage form can be about or approximately 1.0, 1.2, 1.3, 1.4 or 1.5 cc such as amounts which may fall between 1.0 and 1.2, or 1.2 and 1.3, or 1.3 and 1.4, 1.4 and 1.5 cc.

The kit or packaging may include a pre-filled syringe that is ready for immediate delivery to the subject. For example, the packaging system may include a syringe with a leur-lock tip filled with Traumeel, a color coding system (label) for the various dosage units of the Traumeel and size of syringe, a package, a label, and instructions for use.

Since the drug in the syringe is already prepared, the process of drawing up and filtering the drug into a syringe prior to refilling the infusion system is eliminated. Eliminating this process makes filling and refilling the infusion system safer and easier. The pre-filled syringe is easier to use because the practitioner does not have to draw up and filter the drug while administering the therapy to the patient. The syringe's label or plunger is color coded by concentration and syringe sizes, thereby reducing practitioner error and increasing safety to the recipient. The pre-filled syringe also eliminates the potential of contamination of the drug with glass particles from the ampoule, bacteria and the like.

Prefilled syringes can accommodate 1 or multiple dosage units.

Oral dosage forms of Traumeel can be given before, during or after surgery or a surgical procedure. For example, an oral dosage form of Traumeel can be given 3 days prior to surgery or an oral dosage form of Traumeel can be given 2 days prior to surgery or an oral dosage form of Traumeel can be given 1 day prior to surgery or an oral dosage form of Traumeel can be given on the day of surgery or an oral dosage form of Traumeel can be given 1 day after surgery or an oral dosage form of Traumeel can be given 2 days after surgery or an oral dosage form of Traumeel can be given 3 days after surgery.

Use of Traumeel in pill form starting 3 days pre-operatively and continued 3 days post-operatively in conjunction with a single intra-operative injection at the surgical site further decreases post-op bruising, swelling and pain after oral and facial surgery procedures.

Combination therapies can be used with the Traumeel injections. Traumeel combined with analgesics, NSAIDS such as ibuprofen, oxaprozin, carprofen, fenoprofen or fenoprofen calcium, naproxin or naproxin sodium, indoprofen, ketoprofen, flurbiprofen, tolmeti, tolmetin sodium, proquazone, zomepirac, diflunisal and piroxicam, steroids, corticosteroids, Arnica, Traumeel (as an oral, parenteral or topical), aspirin, Tylenol (acetaminophen), COX-2 inhibitors (non-limiting examples include rofecoxib, celecoxib and etoricoxib), flupirtine, paracetamol, opiates and morphinomimetics (non-limiting examples include codeine, oxycodone, hydrocodone, dihydromorphine and pethidine) and any other pain or anti-inflammatory agent know to one of skill in the art are included. Combinations of one or more analgesics is also included. Cold and heat presses can also be used as part of a combination therapy. In one specific instance, Traumeel can be administered topically before, during or after a surgical procedure. In another specific instance, Traumeel can be administered orally before, during or after a surgical procedure.

As used herein a “surgical site” or “surgical area” has its ordinary meaning and means any area or location where there is insult to the skin for example, in a way that allows access to areas protected by the skin. The surgical site can be at any site including a site that is associated with a mucous membrane or mucosal layer of the skin. Preferably, the surgical site is associated with the mouth, oral cavity and/or jaw.

Surgical procedures (“a” or “the” surgery) include any procedure where there is insult such as an incision into the skin of a patient or subject. Preferably, the surgical procedure is done at an area of a mucous membrane. More preferably, the surgical procedure is done in the oral cavity. A non-limiting example of oral surgical procedures includes wisdom teeth removal, dental implants placement, advanced bone reconstruction, the use of BMP (bone morphogenetic proteins) for bone grafting, dental extractions, bone grafting, soft tissue grafting, facial trauma, facial reconstruction, orthognathic surgery, and periapical surgery. Additionally, TMJ (Temporomandibular Joint Disorders) TMD (Temporomandibular Disorders) can be treated with injections into the temporomandibular joints.

The inventive methods of decreasing or reducing post-op swelling, bruising, inflammation and pain after surgery can be practiced in any necessary form of medicine including without limitation, human medicine and veterinary medicine. A patient or subject includes any mammal that has a mucous membrane or that can benefit from this invention. Mammals include without limitation humans, domestic mammals such as, for example, horses, cows, sheep, dogs, pigs, cats and wild mammals, such as for example, tigers, lions, bears, moose, boar primates including monkeys and baboons or other mammals typically found in zoos and also included are laboratory animals such as mice, rats, rabbits, guinea pigs and ferrets.

Without being bound by theory, while reducing acute local inflammation (first phase of adjunct arthritis) in vivo, the preparation did not affect granulocyte function (eg, superoxide anion production and adhesion) or human platelet adhesion in vitro, indicating that the normal defensive and homeostatic functions of these cells are preserved. Traumeel appears to act by regulating the orchestration of the overall process of acute local inflammation rather than by interacting with a specific cell type or biochemical mechanism. Further studies concluded that Traumeel also seems to act by speeding up the healing process instead of blocking edema development from the start.

A decrease or reduction in post-op swelling, bruising, inflammation and pain can be measured visually by a physician. Additionally, anecdotal evidence can be provided by the patient or subject receiving Traumeel, to help determine the amount of post-op swelling, bruising, inflammation and pain experienced. A subjects' experience can be measured with the use of a visual analog scale (VAS) as well as with a 3-dimensional (3D) optical scanner (FaceScan3D; 3D-Shape GmbH, Erlangen, Germany) (Rana et al. Three-Dimensional Evaluation of Postoperative Swelling After Third Molar Surgery Using 2 Different Cooling Therapy Methods: A Randomized Observer-Blind Prospective Study. Journal of Oral and Maxillofacial Surgery Volume 69, Issue 8, Pages 2092-2098, August 2011 which is incorporated herein in its entirety).

The use of Traumeel injection as described herein, reduces overall post-operative surgical swelling, bruising and pain by an average of 50% after oral and facial surgical procedures. The post-operative inflammatory response time shortens with the use of Traumeel by an average of about 20-80%. Often, the post-operative inflammatory response time shortens with the use of Traumeel by an average of about 30-70%. Most often, post-operative inflammatory response time shortens with the use of Traumeel by an average of about 40-60%.

Depending on the type of procedure, post-operative inflammation generally peaks early in the post-operative period. The time to peak post-operative inflammation shortens with the use of Traumeel by an average of about 20-80%. Often, the time to peak post-operative inflammation shortens with the use of Traumeel by an average of about 30-70%. Most often, the time to peak post-operative inflammation shortens with the use of Traumeel by an average of about 40-60%.

Reduction of inflammation includes the reduction of intensity of inflammation as well as the reduction in total time for resolution of inflammation. Generally, after oral surgery and without the use of Traumeel, inflammation peaks at days 3-4. During the peak of inflammation, the inflammation is generally severe. Inflammation at the surgical site is usually resolved in 5-7 days. However, it was unexpectedly found that when Traumeel is administered with surgery, inflammation peaks at day 1 and at that time is moderate. Thereafter, inflammation is resolved by day 3 or day 4.

It is postulated that the use of intramucosal injections of Traumeel can advance wound healing. The reduction in inflammation and the noted recovery times experienced by patients coupled with the T cell stimulatory properties of Traumeel may help facilitate wound healing.

The level of post-operative pain experienced by a subject is reduced with the use of Traumeel injections as described herein. The overall reduction in pain experienced can be reduced by an average of about 20-80%. Often, overall reduction in pain experienced can be reduced by an average of about 30-70%. Most often, the overall reduction in pain experienced can be reduced by an average of about 40-60%.

### Example 1

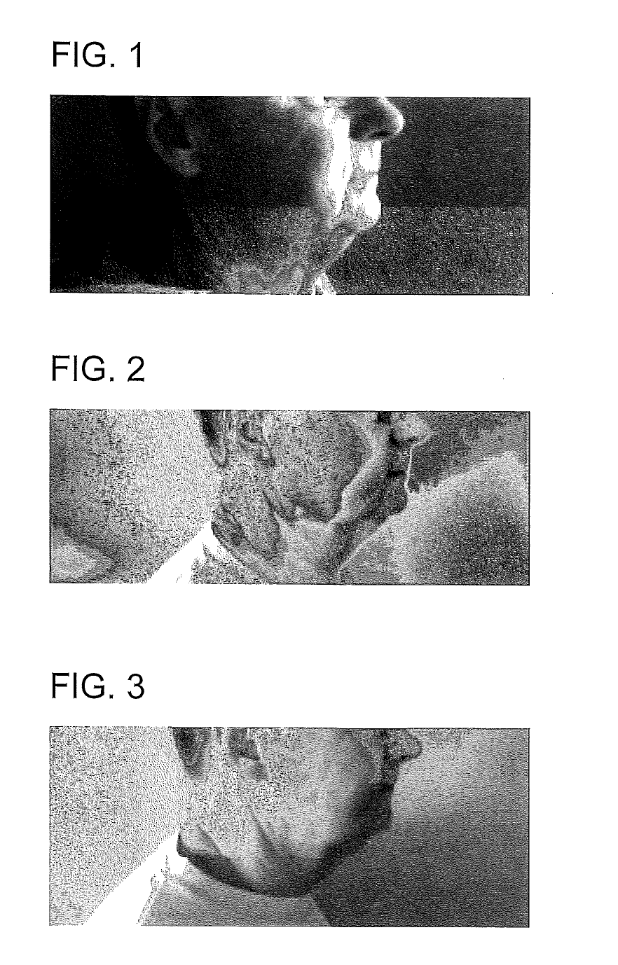

1 cc application of Traumeel was injected submucosally immediately after a sinus lift procedure. The injection was performed in the submucosal fashion at the surgical site. The swelling was significantly smaller then otherwise anticipated due to the injection of Traumeel. FIGS. 1-3 show the post-operative clinical results at 24, 48 and 72 hrs of a patient who underwent a typical sinus lift procedure: at 24 hours post-op, mild swelling is observed and the patient reports no pain; at 48 hours post-op, moderate swelling is observed and the patient reports mild discomfort associated with swelling; at 72 hours post-op, the swelling is almost completely resolved and the patient reports no pain.

### Prophetic Comparative Example 1

A sinus lift procedure is typically associated with moderate to severe post-op swelling and bruising. A sinus lift procedure is performed and no Traumeel is used pre, intra or post-operatively. At 24 hours the patient is reporting moderate swelling, at 48 hours the patient is reporting severe swelling which will last a total of 3-4 days, and at 96 hours the patient is reporting moderate swelling with slight improvement. Usually on day 5-7 post-op, the swelling is 75-85% resolved.

### Prophetic Example 2

1 cc application of Traumeel is injected submucosally immediately after a wisdom tooth extraction procedure. The injection is performed in the submucosal fashion at the surgical site. The swelling is significantly smaller then otherwise anticipated due to the injection of Traumeel. At 24 hours the patient is reporting mild swelling, at 48 hours the patient is reporting moderate swelling, and at 72 hours the patient is reporting mild swelling.

### Prophetic Comparative Example 2

A wisdom tooth extraction is performed and no Traumeel is used pre, intra or post-operatively. At 24 hours the patient is reporting moderate swelling, at 48 hours the patient is reporting severe swelling which will last a total of 3-4 days, after the 96 hour point the patient is reporting moderate swelling with slight improvement. Usually on day 5-7 post-operatively, the swelling is 75-85% resolved.

### Prophetic Example 3

An oral bone reconstruction procedure is performed on a subject. The procedure will be associated with one incision and therefore one surgical site. 0.6 cc of Traumeel is injected intramucosally at one location immediately adjacent the surgical incision and 0.7 cc of submucosal Traumeel is injected at a separate location immediately adjacent the surgical incision. A 3-dimensional (3D) optical scanner will be used to take images of the subject's face and include the area of bone reconstruction. At 24 hours, 85% of peak swelling is measured, at 48 hours, 100% of peak swelling is measured, at 72 hours, 25% of peak swelling is measured, at 96 hours, 10% peak swelling is reached and at 120 hours, 0% of peak swelling is measured.

### Prophetic Comparative Example 3

An oral bone reconstruction procedure is performed on a subject. The procedure will be associated with one incision and therefore one surgical site. No Traumeel is used in this procedure. A 3-dimensional (3D) optical scanner will be used to take images of the subject's face and include the area of bone reconstruction. At 24 hours, 25% of peak swelling is measured, at 48 hours, 50% of peak swelling is measured, at 72 hours, 75% of peak swelling is measured, at 96 hours, 100% peak swelling is reached. At 120 hours, 90% of peak swelling is measured, at 144 hours, 80% of peak swelling is measured, at 168 hours, 70% of peak swelling is measured, at 192 hours, 60% of peak swelling is measured, at 216 hours, 50% of peak swelling is measured, at 240 hours, 40% of peak swelling is measured, at 264 hours, 30% of peak swelling is measured, at 288 hours, 20% of peak swelling is measured, and at 312 hours, 10% of peak swelling is measured.

### Prophetic Example 4

A dental implant procedure is performed on a subject. The procedure will be associated with two surgical sites. 1.2 cc of Traumeel is injected intramucosally at one surgical site immediately adjacent a first surgical incision and 1.3 cc of intramucosal Traumeel is injected at a second surgical site immediately adjacent a second surgical incision. A visual analog scale for pain, graded from 1 to 10 wherein 1 is the least pain and 10 is the greatest, will be used to evaluate the subjects level of pain. At 24 hours, the patient's pain level is at 2, at 48 hours, the patient's pain level is at 3, at 72 hours, the patient's pain level is at 2, at 96 hours, the patient's pain level is at 1.

### Prophetic Comparative Example 4

A dental implant procedure is performed on a subject. The procedure will be associated with two surgical sites. No Traumeel will be used in this procedure. A visual analog scale for pain, graded from 1 to 10 wherein 1 is the least pain and 10 is the greatest, will be used to evaluate the subjects level of pain. At 24 hours, the patient's pain level is at 5, at 48 hours, the patient's pain level is at 9, at 72 hours, the patient's pain level is at 8, at 96 hours, the patient's pain level is at 7, at 120 hours, the patient's pain level is at 6, at 144 hours, the patient's pain level is at 5, at 168 hours, the patient's pain level is at 4, at 192 hours, the patient's pain level is at 3, at 216 hours, the patient's pain level is at 2, and at 240 hours, the patient's pain level is at 1.

### Prophetic Example 5

A patient with a misaligned jaw has orthognathic surgery on day 0. Post-operatively, a 1.0 cc intramucosal injection of Traumeel is administered immediately above the surgical site. Visual inspection of the surgical area at day 1 after the procedure shall reveal moderate swelling. Visual inspection of the surgical area at day 2 after the procedure shall reveal moderate swelling. Visual inspection of the surgical area at day 3 after the procedure shall reveal at least a 50% reduction in swelling as compared to the amount of swelling during peak swelling.

### Prophetic Comparative Example 5

A patient with a misaligned jaw has orthognathic surgery on day 0. No Traumeel is administered during or after this surgical procedure. Visual inspection of the surgical area at day 1 after the procedure shall reveal moderate swelling. Visual inspection of the surgical area at day 2 after the procedure shall reveal severe swelling. Visual inspection of the surgical area at day 3 after the procedure shall reveal severe swelling. Visual inspection of the surgical area at day 4 after the procedure shall reveal severe swelling. Visual inspection of the surgical area at day 5 after the procedure shall reveal moderate swelling. Visual inspection of the surgical area at day 6 after the procedure shall reveal moderate swelling. Visual inspection of the surgical area at day 7 after the procedure shall reveal moderate swelling. Visual inspection of the surgical area at day 8 after the procedure shall reveal moderate swelling. Visual inspection of the surgical area at day 9 after the procedure shall reveal mild swelling.

### Prophetic Comparative Example 5.1

A patient with a misaligned jaw has orthognathic surgery on day 0. Post-operatively, a 3.5 cc intramucosal injection of Traumeel is administered immediately above the surgical site. Visual inspection of the surgical area at day 1 after the procedure shall reveal moderate swelling. Visual inspection of the surgical area at day 2 after the procedure shall reveal severe swelling. Visual inspection of the surgical area at day 3 after the procedure shall reveal severe swelling. Visual inspection of the surgical area at day 4 after the procedure shall reveal severe swelling. Visual inspection of the surgical area at day 5 after the procedure shall reveal moderate swelling. Visual inspection of the surgical area at day 6 after the procedure shall reveal moderate swelling. Visual inspection of the surgical area at day 7 after the procedure shall reveal moderate swelling. Visual inspection of the surgical area at day 8 after the procedure shall reveal moderate swelling. Visual inspection of the surgical area at day 9 after the procedure shall reveal mild swelling.

### Example 6

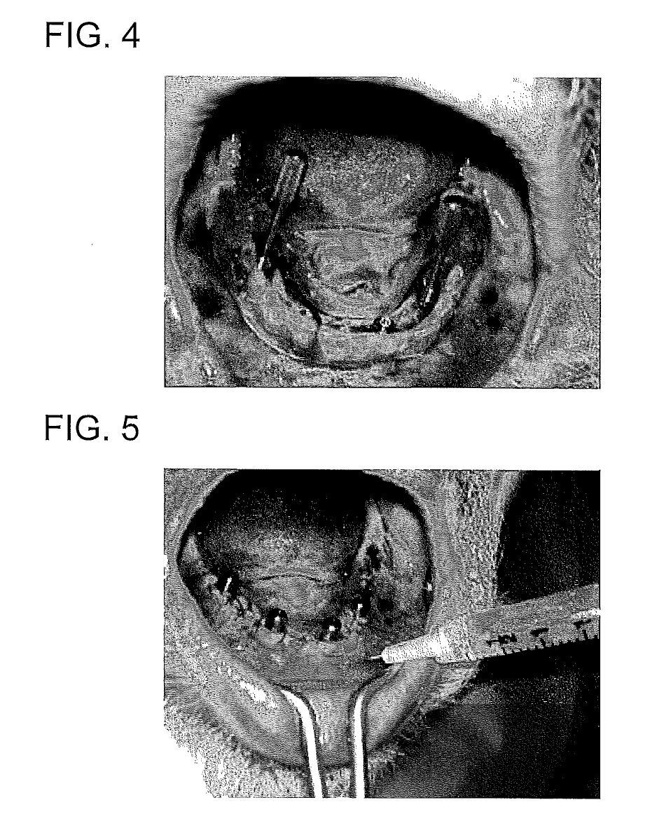

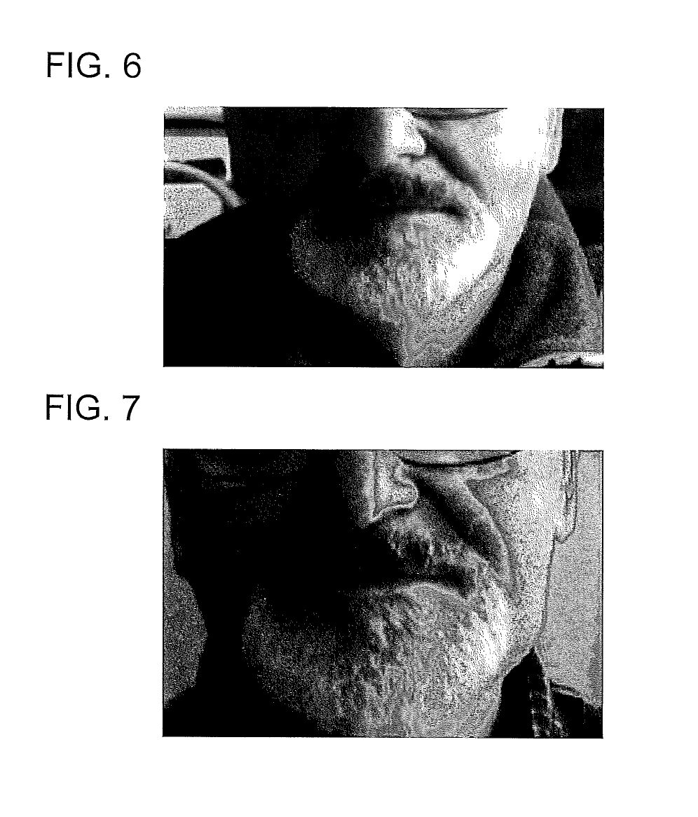

FIG. 4 shows a patient undergoing multiple implants placement. In this FIG. 4, a full thickness mucoperiosteal flap is reflected and extensive osseous surgery is performed. FIG. 5 shows a 1.1 cc of Traumeel injected in the submucosal fashion on the right side (in close proximity to one location of the surgical area), and 1.1 cc on the left side. The total volume of Traumeel injected at this large surgical area was 2.2 cc (one full Ampule). FIG. 6 shows the patient 24 hours after the surgery where he is exhibiting mild to moderate swelling around the chin and lower jaw areas. FIG. 7 shows the patient 48 hours post-op and shows his swelling has almost completely resolved (his swelling is 75-80% resolved).

### Prophetic Comparative Example 6

A patient will undergo multiple implants replacement surgery. No Traumeel is administered. The amount of post-op swelling usually anticipated with this procedure is moderate to severe. The peak swelling and bruising is expected to be 3-4 days post-operatively and complete resolution is anticipated 6-7 days post-operative.

### Example 7

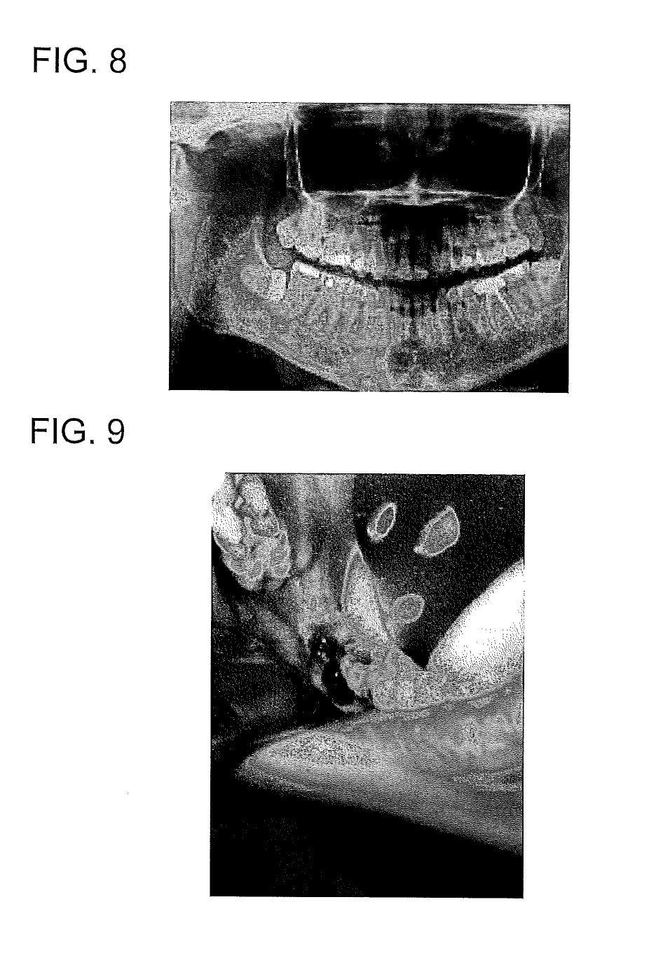

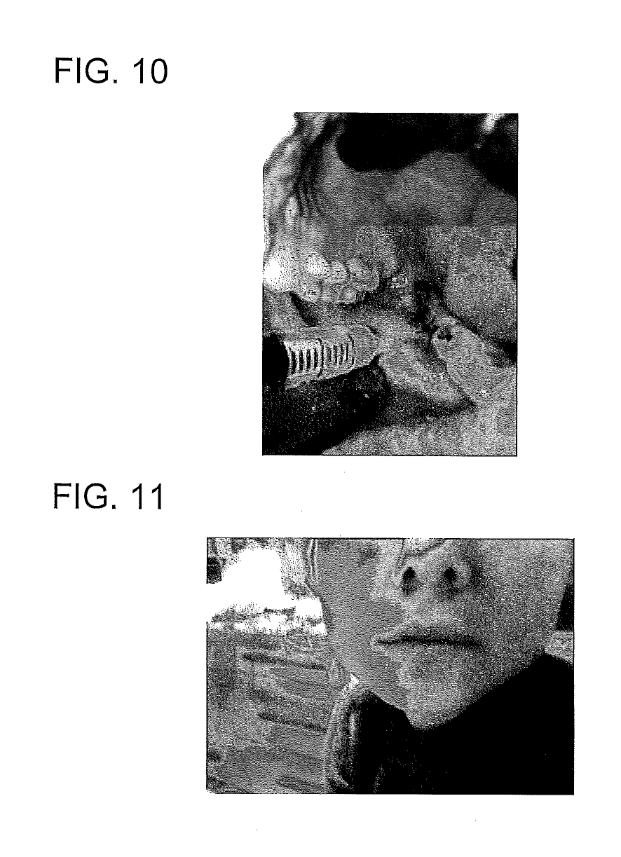

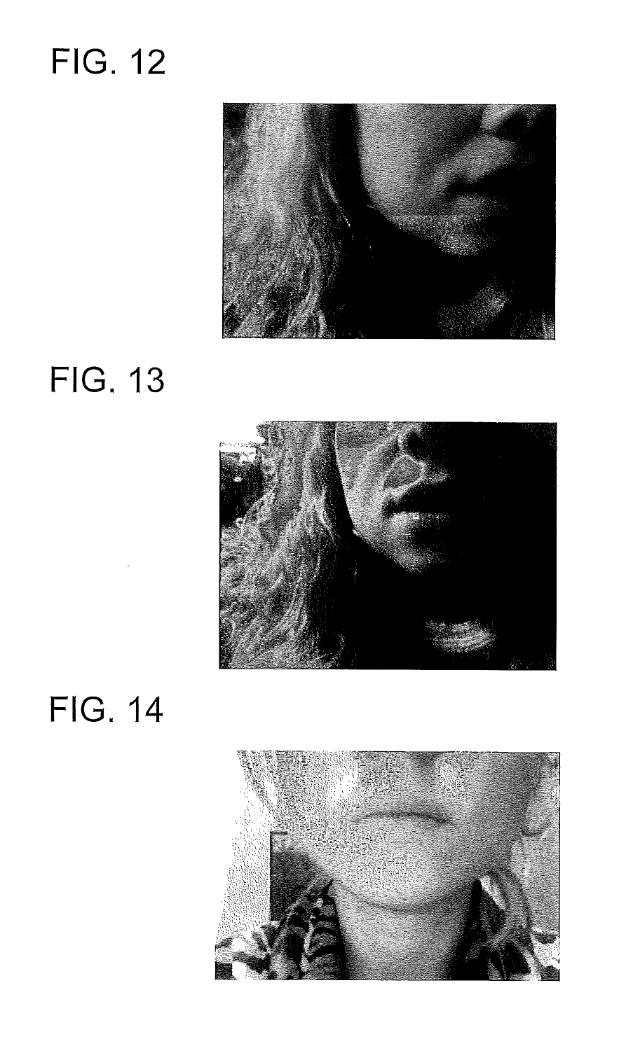

FIG. 8 shows a full bony impacted wisdom tooth (Tooth #32 (lower R). FIG. 9, taken intra-operative, shows the surgical removal required a full thickness mucoperiosteal flap, and partial removal of bone. FIG. 10, shows 1 cc of Traumeel injected intramucosally over the surgical site. FIG. 11 shows the patient 24 hours post-operatively where mild to moderate swelling is observed. FIGS. 12-13 both show the patient's swelling and bruising 48 hours after the surgical procedure. Moderate amount of swelling is observed. In FIG. 14, the patient is 72 hours post-operative. At 72 hours after the surgical procedure, the swelling is 75-85% resolved and the patient is feeling back to normal.

### Prophetic Comparative Example 7

A full bony impacted wisdom tooth (Tooth #32 (lower R) is to be extracted. No Traumeel is administered. The amount of post-op swelling usually anticipated with this procedure is moderate to severe. The peak swelling and bruising is expected to be 3-4 days post-operatively and complete resolution is anticipated 6-7 days post-operative.

### Example 8

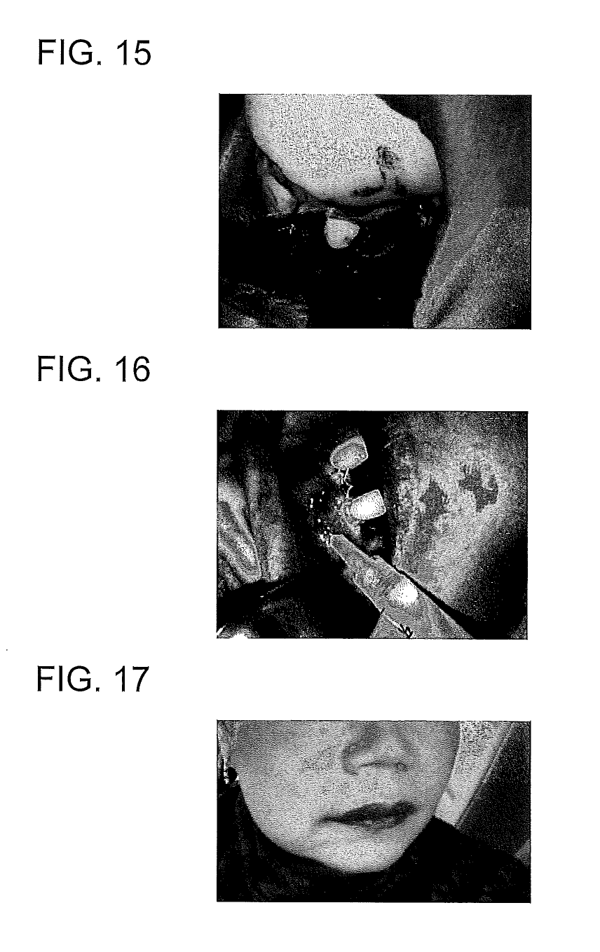

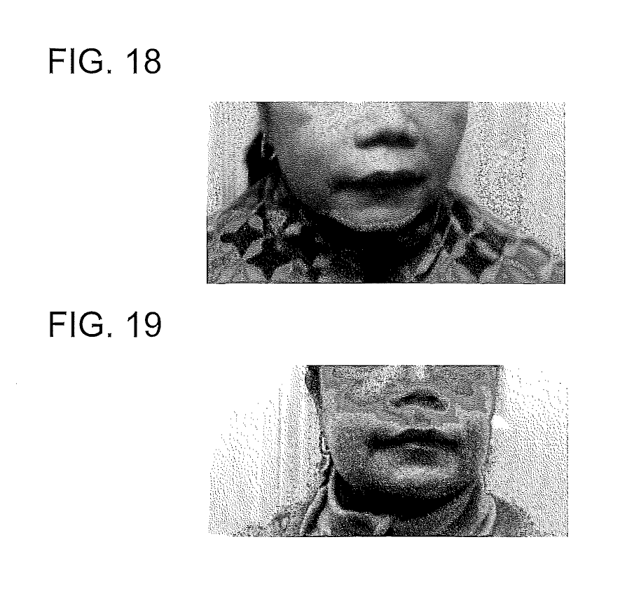

FIG. 15 shows an extensive bone grafting performed in the anterior maxilla, extending to the posterior maxillary region. A full thickness mucoperiosteal flap is reflected, and significant tissue undermining has been performed to achieve primary closure. FIG. 16 shows a total of 2 cc of Traumeel injected into the mucosal tissue. 1 cc of Traumeel is injected into the mucosal tissue anteriorly and 1 cc of Traumeel is injected into the mucosal tissue posteriorly. FIG. 17 shows the patient 24 hours post-operative and shows a mild to moderate amount of swelling. FIG. 18 shows the patient at 48 hours post-op. The swelling has peaked and some bruising is visible. At the 48 hour mark, the patient reports mild to moderate discomfort. FIG. 19 shows the patient 72 hours post-op. The swelling is 75% resolved and mild discomfort is reported by patient.

### Prophetic Comparative Example 8

An extensive bone grafting is performed in the anterior maxilla, extending to the posterior maxillary region. No Traumeel is administered. The expected post-op swelling is usually moderate to severe with the pick of swelling and bruising on post-op day 4, and 75% to complete resolution expected on post-op day 7.

### Prophetic Example 9

1.5 to 2 cc of Traumeel is injected into each temporomandibular joint.

For the relief of pain and discomfort, Traumeel can be injected intrajoint. A dosage unit of Traumeel can be from 0.5 to 2 cc. Preferably, the dosage unit is from 1.0 to 1.5 cc. For example, the dosage form can be 1.0, 1.2, 1.3, 1.4 or 1.5 cc. Also, the dosage form can be about or approximately 1.0, 1.2, 1.3, 1.4 or 1.5 cc such as amounts which may fall between 1.0 and 1.2, or 1.2 and 1.3, or 1.3 and 1.4, 1.4 and 1.5 cc.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

It is contemplated that numerical values, as well as other values that are recited herein are modified by the term “about”, whether expressly stated or inherently derived by the discussion of the present disclosure. As used herein, the term “about” defines the numerical boundaries of the modified values so as to include, but not be limited to, tolerances and values up to, and including the numerical value so modified. That is, numerical values can include the actual value that is expressly stated, as well as other values that are, or can be, the decimal, fractional, or other multiple of the actual value indicated, and/or described in the disclosure.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I claim:

1. A method of reducing or ameliorating pain, inflammation or bruising at a surgical site comprising injecting Traumeel at the surgical site wherein the Traumeel is injected in the mucosal layer of the skin.

2. The method according to claim 1, wherein the dosage unit of Traumeel is from 0.5 to 2 cc.

3. The method according to claim 2, wherein the dosage unit of Traumeel is from 1 to 1.5 cc.

4. The method according to claim 1, wherein the dosage unit of Traumeel is 0.5 to 2 cc and said dosage unit is administered once for every 0.5-3 cm² area of the surgical site.

5. The method according to claim 1, wherein the surgical site is in the oral cavity.

6. The method according to claim 1, wherein the surgical site is the result of a surgery selected from the group consisting of: wisdom teeth removal, dental implants placement, advanced bone reconstruction, the use of BMP (bone morphogenetic proteins), dental extractions, bone grafting, soft tissue grafting, facial trauma, facial reconstruction, orthognathic surgery, periapical surgery and any other oral surgical procedure.

7. The method according to claim 1, wherein one or more additional therapeutic agents are administered before, during and/or after a surgical procedure and the additional therapeutic agents are agents known to reduce pain, inflammation and/or bruising.

8. A method of reducing the time to maximum swelling around a surgical site, comprising intra-operatively or post-operatively injecting Traumeel at the surgical site.

9. The method according to claim 7, wherein the Traumeel is injected into the mucosal layer.

10. The method according to claim 7, wherein the dosage unit of Traumeel is from 0.5 to 2 cc.

11. The method according to claim 9, wherein the dosage unit of Traumeel is from 1 to 1.5 cc.

12. The method according to claim 7, wherein the surgical site is in the oral cavity.

13. A method of reducing the total inflammatory response time at a surgical site post-operatively comprising injecting Traumeel at the surgical site wherein the Traumeel is injected in the mucosal layer of the epidermis.

14. The method according to claim 12, wherein the dosage unit of Traumeel is from 0.5 to 2 cc.

15. The method according to claim 13, wherein the dosage unit of Traumeel is from 1 to 1.5 cc.

16. The method according to claim 13, wherein the surgical site is in the oral cavity.

17. A method of inhibiting pro-inflammatory cytokines while stimulating anti-inflammatory cytokines at a surgical site comprising injecting Traumeel at the surgical site wherein the Traumeel is injected into the mucosal layer of the epidermis.

18. The method according to claim 16, wherein the dosage unit of Traumeel is from 0.5 to 2 cc.

19. A method of treating TMJ (Temporomandibular Joint Disorders) or TMD (Temporomandibular Disorders) comprising injecting 1 to 1.5 cc of Traumeel into the Temporomandibular joint.

20. A dosage unit form of Traumeel, wherein the dosage unit is from 0.5 to 2 cc and is used according to claim 1.
